(19) United States
(12) Patent Application Publication
(10) Pub. No.: US 2002/0168679 A1
(43) Pub. Date: Nov. 14, 2002

(54) STAINING AGENTS AND PROTOCOLS FOR CHARACTERIZING MALIGNANT CELLS IN CULTURE

(76) Inventors: GREGORY J. NAUS, OAKMONT, PA (US); PAUL L. KORNBLITH, PITTSBURGH, PA (US); DENNIS R. BURHOLT, PITTSBURGH, PA (US); MICHAEL P. MEYER, CARNEGIE, PA (US)

Correspondence Address: BARBARA E JOHNSON, 700 KOPPERS BUILDING, 436 SEVENTH AVENUE, PITTSBURG, PA, 152191818

(21) Appl. No.: 09/095,993

(22) Filed: Jun. 11, 1998

(51) Int. Cl. G01N 33/53; C07K 16/00

(52) U.S. Cl. 435/7.1; 530/387.1

(57) ABSTRACT

An improved system for screening a multiple of candidate therapeutic or chemotherapeutic agents for efficacy as to a specific patient, in which a tissue sample from the patient is harvested, cultured and separately exposed to a plurality of treatments and/or therapeutic agents for the purpose of objectively identifying the best treatment or agent for the particular patient. Specific method innovations such as tissue sample preparation techniques render this method practically as well as theoretically useful. The identity of the malignant cells in culture is advantageously confirmed using binding reagents/staining systems specific for epithelial cells, since carcinomas are ubiquitously epithelial in nature. Cells of interest and thus confirmed as epithelial/carcinomal may then be assayed for sensitive to an infinite variety of malignancy treating agents including chemotherapeutic agents, radiation, immunotherapy, and so on.

### FIELD OF THE INVENTION

The invention relates to screening and testing of active agents, including chemotherapeutic agents, to predict potential efficacy in individual patients in whom treatment with such agents is indicated, and staining compositions and protocols to confirm the identity of the cultured malignant cells of interest.

### INTRODUCTION

All active agents including chemotherapeutic active agents are subjected to rigorous testing as to efficacy and safety prior to approval for medical use in the U.S. Methods of assessing efficacy have included elaborate investigations of large populations in double blind studies as to a given treatment method and/or active agent, with concomitant statistical interpretation of the resulting data, but these conclusions are inevitably generalized as to patient populations taken as a whole. In many pharmaceutical disciplines and particularly in the area of chemotherapy, however, the results of individual patient therapy may not comport with generalized data—to the detriment of the individual patient. The need has been long recognized for a method of assessing the therapeutic potential of active agents, including but not limited to chemotherapeutic agents, for their efficacy as to a given individual patient, prior to the treatment of that patient.

Prior art assays already exist which expose malignant tissue of various types to a plurality of active agents, for the purpose of assessing the best choice for therapeutic administration. For example, in Kruczynski, A., et al., “Evidence of a direct relationship between the increase in the in vitro passage number of human non-small-cell-lung cancer primocultures and their chemosensitivity,” Anticancer Research, vol. 13, no. 2, pp. 507-513 (1993), chemosensitivity of non-small-cell-lung cancers was investigated in in vivo grafts, in in vitro primocultures and in commercially available long-term cancer cell lines. The increase in chemosensitivity was documented and correlated with morphological changes in the cells in question. Sometimes animal model malignant cells and/or established cell cultures are tested with prospective therapy agents, see, for example, Arnold, J. T., “Evaluation of chemopreventive agents in different mechanistic classes using a rat tracheal epithelial cell culture transformation assay,” Cancer Res.., vol. 55, no. 3, pp. 537-543 (1995).

When actual patient cells are used to form in vitro assays focussed on individual patients, in typical prior art processes the cells are harvested (biopsied) and trypsinized (connective tissue digested with the enzyme trypsin) to yield a cell suspension suitable for conversion to the desired tissue culture form. The in vitro tissue culture cell collections which result from these techniques are generally plagued by their inability accurately to imitate the chemosensitivity of the original tumor or other cell biopsy. Standard cloning and tissue culture techniques are moreover excessively complicated and expensive for use in a patient-by-patient assay setting. A need thus remains for a technique of tissue culture preparation which provides cell cultures, for drug screening purposes, in which after simple preparation the cell cultures react in a manner equivalent to their in vivo reactivity, to enable drug or chemotherapeutic agent screening as to a particular patient for whom such screening is indicated.

### SUMMARY OF THE INVENTION

In order to meet this need, the present invention is an improved system for screening a multiple of candidate therapeutic or chemotherapeutic agents for efficacy as to a specific patient and for confirming the identity of the malignant cells being tested, in which a tissue sample from the patient is harvested, cultured and separately exposed to a plurality of treatments and/or therapeutic agents for the purpose of objectively identifying the best treatment for the cultured cells obtained from the patient. Specific method innovations such as tissue sample preparation techniques render this method practically as well as theoretically useful. One particularly important tissue sample preparation technique is the initial preparation of cohesive multicellular particulates of the tissue sample, rather than enzymatically dissociated cell suspensions or preparations, for initial tissue culture monolayer preparation. With respect to the culturing of malignant cells, for example, it is believed (without any intention of being bound by the theory) that by maintaining the malignant cells within a multicellular particulate of the originating tissue, growth of the malignant cells themselves is facilitated versus the overgrowth of fibroblasts or other cells which tends to occur when suspended tumor cells are grown in culture. Practical monolayers of cells may thus be formed to enable meaningful screening of a plurality of treatments and/or agents. Growth of cells is monitored to ascertain the time to initiate the assay and to determine the growth rate of the cultured cells; sequence and timing of drug addition is also monitored and optimized. By subjecting uniform samples of cells to a wide variety of active agents (and concentrations thereof), the most efficacious agent can be determined. For assays concerning cancer treatment, a two-stage evaluation is contemplated in which both acute cytotoxic and longer term inhibitory effect of a given anti-cancer agent are investigated.

As an important part of the above technique, staining compositions and protocols are used (1) to identify whether the malignant cells grown in culture are epithelial cells and, if not, (2) to confirm that the cells grown in culture are specifically non-epithelial. The overall method, including the method of characterizing the cells grown in culture with these staining compositions and protocols as well as unique antibody cocktails therefor, forms the core of the subject matter of this continuation-in-part specification.

### DETAILED DESCRIPTION OF THE INVENTION

The present invention is a system for screening a multiple of candidate therapeutic or chemotherapeutic agents for efficacy as to a specific patient, in which a tissue sample from the patient is harvested and separately exposed to a plurality of treatments and/or therapeutic agents for the purpose of objectively identifying the best treatment or agent. Specific method innovations such as tissue sample preparation techniques render this method practically as well as theoretically useful. One particularly important tissue sample preparation technique is the initial preparation of cohesive multicellular particulates (explants) of the tissue sample, rather than enzymatically dissociated cell suspensions or preparations, for initial tissue culture monolayer preparation. Cell growth, and sequence and timing of drug addition, are monitored and optimized.

As an important part of the above technique, staining compositions and protocols are used (1) to identify whether the malignant cells grown in culture are epithelial cells and, if not, (2) to confirm that the cells grown in culture are specifically non-epithelial. The overall method, including the method of characterizing the cells grown in culture with these staining compositions and protocols as well as unique antibody cocktails therefor, forms the core of the subject matter of this continuation-in-part specification.

An important application of the present invention is the screening of chemotherapeutic agents and other antineoplastic therapies against tissue culture preparations of malignant cells from the patients from whom malignant samples are biopsied. Related anti-cancer therapies which can be screened using the inventive system are both radiation therapy and agents which enhance the cytotoxicity of radiation, as well as immunotherapeutic anti-cancer agents. Screening processes for treatments or therapeutic agents for nonmalignant syndromes are also embraced within this invention, however, and include without limitation agents which combat hyperproliferative syndromes, such as psoriasis, or wound healing agents. Nor is the present efficacy assay limited only to the screening of active agents which speed up (healing) or slow down (anti-cancer, anti-hyperproliferative) cell growth because agents intended to enhance or to subdue intracellular biochemical functions may be tested in the present tissue culture system also. For example, the formation or blocking of enzymes, neurotransmitters and other biochemicals may be screened with the present assay methods prior to treatment of the patient.

When the patient is to be treated for the presence of tumor, in the preferred embodiment of the present invention a tumor biopsy of >100 mg of non-necrotic, non-contaminated tissue is harvested from the patient by any suitable biopsy or surgical procedure known in the art. Biopsy sample preparation generally proceeds as follows under a Laminar Flow Hood which should be turned on at least 20 minutes before use. Reagent grade ethanol is used to wipe down the surface of the hood prior to beginning the sample preparation. The tumor is then removed, under sterile conditions, from the shipping container and is minced with sterile scissors. If the specimen arrives already minced, the individual tumor pieces should be divided into four groups. Using sterile forceps, each undivided tissue quarter is then placed in 3 ml sterile growth medium (Standard F-10 medium containing 17% calf serum and a standard amount of Penicillin and Streptomycin) and systematically minced by using two sterile scalpels in a scissor-like motion, or mechanically equivalent manual or automated opposing incisor blades. This cross-cutting motion is important because the technique creates smooth cut edges on the resulting tumor multicellular particulates. Preferably but not necessarily, the tumor particulates each measure 1 mm³. After each tumor quarter has been minced, the particles are plated in culture flasks using sterile pasteur pipettes (9 explants per T-25 or 20 particulates per T-75 flask). Each flask is then labeled with the patient's code, the date of explanation and any other distinguishing data. The explants should be evenly distributed across the bottom surface of the flask, with initial inverted incubation in a 37° C. incubator for 5-10 minutes, followed by addition of about 5-10 ml sterile growth medium and further incubation in the normal, non-inverted position. Flasks are placed in a 35° C., non-CO₂ incubator. Flasks should be checked daily for growth and contamination. Over a period of a few weeks, with weekly removal and replacement of 5 ml of growth medium, the explants will foster growth of cells into a monolayer. With respect to the culturing of malignant cells, it is believed (without any intention of being bound by the theory) that by maintaining the malignant cells within a multicellular particulate of the originating tissue, growth of the malignant cells themselves is facilitated versus the overgrowth of fibroblasts (or other unwanted cells) which tends to occur when suspended tumor cells are grown in culture.

The use of the above procedure to form a cell monolayer culture maximizes the growth of malignant cells (and only malignant cells) from the tissue sample, and thus optimizes ensuing tissue culture assay of chemotherapeutic action of various agents to be tested. Enhanced growth of actual malignant cells is only one aspect of the present invention, however; another important feature is the growth rate monitoring system used to oversee growth of the monolayer once formed. Once a primary culture and its derived secondary monolayer tissue culture has been initiated, the growth of the cells is monitored to ascertain the time to initiate the chemotherapy assay and to determine the growth rate of the cultured cells.

Monitoring of the growth of cells is conducted by counting the cells in the monolayer on a periodic basis, without killing or staining the cells and without removing any cells from the culture flask. The counting may be done visually or by automated methods, either with or without the use of estimating techniques known in the art (counting in a representative area of a grid multiplied by number of grid areas, for example). Data from periodic counting is then used to determine growth rates which may or may not be considered parallel to growth rates of the same cells in vivo in the patient. If growth rate cycles can be documented, for example, then dosing of certain active agents can be customized for the patient. The same growth rate can be used to evaluate radiation treatment periodicity, as well. It should be noted that with the growth rate determinations conducted while the monolayers grow in their flasks, the present method requires no hemocytometry, flow cytometry or use of microscope slides and staining, with all their concomitant labor and cost.

Protocols for monolayer growth rate generally use a phase-contrast inverted microscope to examine culture flasks incubated in a 37° C. (5% CO₂) incubator. When the flask is placed under the phase-contrast inverted microscope, ten fields (areas on a grid inherent to the flask) are examined using the 10x objective, with the proviso that the ten fields should be non-contiguous, or significantly removed from one another, so that the ten fields are a representative sampling of the whole flask. Percentage cell occupancy for each field examined is noted, and averaging of these percentages then provides an estimate of overall percent confluency in the cell culture. When patient samples have been divided between two or among three or more flasks, an average cell count for the total patient sample should be calculated. The calculated average percent confluency should be entered into a process log to enable compilation of data—and plotting of growth curves—over time. Monolayer cultures may be photographed to document cell morphology and culture growth patterns. The applicable formula is:

$$Percent confluency = \frac{estimate of the area occupied by cells}{total area in an observed field}$$

As an example, therefore, if the estimate of area occupied by the cells is 30% and the total area of the field is 100%, percent confluency is 30/100, or 30.

Adaptation of the above protocol for non-tumor cells is straightforward and generally constitutes an equivalent procedure.

Active agent screening using the cultured cells does not proceed in the initial incubation flask, but generally proceeds using plates such as microtiter plates. The performance of the chemosensitivity assay used for screening purposes depends on the ability to deliver a reproducible cell number to each row in a plate and/or a series of plates, as well as the ability to achieve an even distribution of cells throughout a given well. The following procedure assures that cells are reproducibly transferred from flask to microtiter plates, and cells are evenly distributed across the surface of each well.

The first step in preparing the microtiter plates is, of course, preparing and monitoring the monolayer as described above. The following protocol is exemplary and susceptible of variation as will be apparent to one skilled in the art. Cells are removed from the culture flask and a cell pellet is prepared by centrifugation. The cell pellet derived from the monolayer is then suspended in 5ml of the growth medium and mixed in a conical tube with a vortex for 6 to 10 seconds. The tube is then rocked back and forth 10 times. A 36μl droplet from the center of the conical tube is pipetted onto one well of a 96 well plate. A fresh pipette is then used to pipette a 36 μl aliquot of trypan blue solution, which is added to the same well, and the two droplets are mixed with repeated pipette aspiration. The resulting admixture is then divided between two hemocytometer chambers for examination using a standard light microscope. Cells are counted in two out of four hemocytometer quadrants, under 10x magnification. Only those cells which have not taken up the trypan blue dye are counted. This process is repeated for the second counting chamber. An average cell count per chamber is thus determined. Using means known in the art, the quadrant count values are checked, logged, multiplied by 10⁴to give cells/ml, and the total amount of fluid (growth medium) necessary to suspend remaining cell aliquots is calculated accordingly.

After the desired concentration of cells in medium has been determined, additional cell aliquots from the monolayer are suspended in growth medium via vortex and rocking and loaded into a Terasaki dispenser known in the art. Aliquots of the prepared cell suspension are delivered into the microtiter plates using Terasaki dispenser techniques known in the art. A plurality of plates may be prepared from a single cell suspension as needed. Plates are then wrapped in sterile wet cotton gauze and incubated in an incubator box by means known in the art.

After the microtiter plates have been prepared, exposure of the cells therein to active agent is conducted according to the following exemplary protocol. During this portion of the inventive assay, the appropriate amount of specific active agent is transferred into the microtiter plates prepared as described above. A general protocol, which may be adapted, follows. Each microtiter plate is unwrapped from its wet cotton gauze sponge and microscopically examined for cell adhesion. Control solution is dispensed into delineated rows of wells within the grid in the microtiter plate, and appropriate aliquots of active agent to be tested are added to the remaining wells in the remaining rows. Ordinarily, sequentially increasing concentrations of the active agent being tested are administered into progressively higher numbered rows in the plate. The plates are then rewrapped in their gauze and incubated in an incubator box at 37° C. under 5% CO₂. After a predefined exposure time, the plates are unwrapped, blotted with sterile gauze to remove the agent, washed with Hank's Balance Salt Solution, flooded with growth medium, and replaced in the incubator in an incubator box for a predefined time period, after which the plates may be fixed and stained for evaluation.

Fixing and staining may be conducted according to a number of suitable procedures; the following is representative. After removal of the plates from the incubator box, culture medium is poured off and the plates are flooded with Hank's Balance Salt Solution. After repeated flooding (with agitation each time) the plates are then flooded with reagent grade ethanol for 2-5 minutes. The ethanol is then poured off. Staining is accomplished with approximately 5 ml of Giemsa Stain per plate, although volume is not critical and flooding is the goal. Giemsa stain should be left in place 5 min.±30 seconds as timing influences staining intensity. The Giemsa stain is then poured off and the plates are dipped 3 times in cold tap water in a beaker. The plates are then inverted, shaken vigorously, and air dried overnight (with plate lids off) on a rack on a laboratory bench. Cells per well are then counted manually or by automated and/or computerized means, to derive data regarding chemosensitivity of cells at various concentrations of exposure. One particularly useful computer operating environment for counting cells is the commercially available OPTIMATE compiler, which is designed to permit an optical counting function well suited to computerized cell counting procedures and subsequent calculations.

The above procedures do not change appreciably when cell growth promoters are assayed rather than cell arresting agents such as chemotherapeutic agents. The present assay allows cell death or cell growth to be monitored with equal ease. In any case, optimization of use of the present system will involve the comparative testing of a variety of candidate active agents, for selection of the best candidate for patient treatment based upon the in vitro results. One particularly advantageous embodiment of the above described invention comprises a two-stage assay for cytotoxicity followed by evaluation of longer-term inhibitory effect. Chemotherapeutic agents may thus be evaluated separately for both their direct chemotherapeutic effect as well as for their longer duration efficacy.

Identification of one or more active agents or chemotherapeutic agents is peripheral to the present invention, which is intended for the efficacy screening of any or all of them as to a given patient. Literally any active agent may be screened according to the present invention; listing exemplary active agents is thus omitted here.

The essence of the invention thus includes the important feature of the simplicity of the present system. Cohesive multicellular particulates of the patient tissue to be tested are used to form cell monolayers. Growth of those monolayers is in turn monitored for accurate prediction of correlating growth of the same cells in vivo. Finally, differing concentrations of a number of active agents may be tested for the purpose of determining not only the most appropriate agent but the most appropriate concentration of that agent for actual patient exposure (according to the calculated cell growth rates) . It is also important to note, in the context of the invention, that the present system allows in vitro tests to be conducted in suspensions of tissue culture monolayers grown in nutrient medium under fast conditions (a matter of weeks), rather than with single cell progeny produced by dilution cloning over long periods of time. In some cases, the present invention is a two stage assay for both cytotoxicity and the longer-term growth inhibitory.

It has now been determined that, despite the reliability of the above-disclosed technique to grow out only the cells of interest (malignant cells), it is additionally possible to increase the value of the associated assay with the use of staining compositions and protocols designed to characterize the malignant cells thus grown. In other words, the tissue preparation and cell culturing technique itself offers a first assurance that the cells grown out of the tumor are really the malignant tumor cells and not fibroblasts or other nonmalignant cells of no diagnostic value. As a separate confirmation, the present staining compositions and protocols offer a second, independent assurance that the cells subject to diagnostic or prognostic assay are in fact malignant cells in culture. One important characterization has to do with the nature of the malignant cells as epithelial, which is in turn an indicator of the carcinoma type of malignancy. Other characterizations of malignant cells are intended to fall within the scope of the present invention as well, although the characterization of the cells as epithelial or not is of primary importance.

The technique is practiced as follows. The same cell culturing and well distribution process is used as in the cytotoxicity assay described above, but rather than exposing the cells to chemotherapeutic or other agents, the cells are instead fixed and stained. With the stain or stain cocktail described below, the epithelial cells are identified by their intermediate filaments and/or specific membrane antigens by means of a monoclonal antibody immunoperoxidase technique. The fixative used can be any fixative which does not alter the cellular molecular markers of interest. The fixed, stained cells are then counted. If the specimen is positive for epithelial cells, the process is complete. If the specimen is negative for epithelial cells, an independent fixing and staining process is subsequently completed, with fresh cells from identical wells, using Vimentin as a stain to confirm the non-epithelial nature of the cells.

The importance of having a stain or stain cocktail (i.e., antibody cocktail), as well as an overall protocol, for identifying epithelial cells in biopsies of malignant tumors is as follows. In the basic cytotoxicity assay, the tissue culture technique is designed to grow out the cells of the tumor of origin and in fact consistently does so. Despite such reliable predictability, however, the fact that the cells of the tumor of origin did in fact grow out, and not fibroblasts or other cells, must be confirmed with independent proof before the cells can be used with complete assurance in the appropriate patient assay(s). The present technology provides a means to obtain this confirmation, which in turn furthers the interests of good laboratory and medical practice.

As a general consideration, the staining compounds or compositions of interest for use in the present technology are those which bind with cellular molecular markers unique either to epithelial or to non-epithelial cells. The invention inheres in the following two aspects: the improvement of the cytotoxicity assay by adding the epithelial staining protocol with any known epithelial stain; and the further improvement wherein specially designed stain cocktails maximize the likelihood that the presence of any known intermediate filament or specific membrane antigen, characteristic of epithelial cells, will be identified if present.

Many carcinomas are positive for any one of the intermediate filaments or specific membrane antigens characteristic of epithelial cells; virtually all if not all carcinomas are positive for one of a number of such intermediate filaments or specific membrane antigens. For example, “epithelial membrane antigen” (“EMA”) glycoproteins are known in the art and can be bound with various antiepithelial membrane antigen antibodies including monoclonal antibodies. Cytokeratin is another important epithelial cell marker and binding reagents including monoclonal antibodies are available which are specific to cytokeratin. While antisera can be raised in vivo against markers such as EMA glycoproteins and cytokeratin, as a practical matter commercially available polyclonal or monoclonal antibodies are used in the following protocols, with monoclonal antibodies being preferred.

Binding of the epithelial marker is revealed with associated staining procedures and reactions which give a visual indication that the marker binding took place. Those skilled in the art already appreciate various techniques already available—in the general field of “immunocytochemistry”—to reveal antibody-antigen reactions. One known way to accomplish this visualization when antibody binding reagents are used is with the “labeled streptavidin procedure”. In this procedure, after the specimen is exposed to antibodies specific to the target antigen, a secondary “link” antibody is added. The secondary biotinylated “link” antibody consists of anti-mouse and anti-rabbit antibodies which bind universally to most primary monoclonal or polyclonal antibodies. The “link” will also connect to the tertiary reagent (peroxidase-labeled streptavidin) through chemical bonding between the biotin on the secondary reagent and the streptavidin on the streptavidin/peroxidase conjugate. Staining is completed by incubating the specimen and primary, secondary and tertiary agents in the presence of a chromagen, so that the peroxidase and the chromagen form a visible precipitate. Alternatively, a fluorescein-based detection system can be used to visualize the primary antibody, or a third alternative known in the art as the digoxigenin-conjugated detection system may be used.

Of the various epithelial markers, three have received the most widespread attention in the literature: EMA glycoproteins, cytokeratin, and carcinoembryonic antigen. In the context of this invention, the first two are the most important because literally any epithelial cell will have at least either one EMA glycoprotein on the surface thereof or a cytokeratin intermediate filament present. Therefore, the present invention resides not only in binding and staining for an epithelial marker on the surfaces of the specimen cells, but in simultaneously assaying for either or both of EMA glycoprotein(s) and cytokeratin. The cocktails of the present invention therefore contain binding reagents for both EMA glycoproteins and cytokeratin and, importantly, are selected to include the most generally applicable binding reagents in combination so that the cocktail has the broadest binding scope possible. The cocktails identified in Examples 1 and 2, for example, represent a combination of two general binding reagents (containing a total of three monoclonal antibodies) for cytokeratin, admixed with a general binding reagent for EMA glycoprotein. The dual benefit of this admixture of general binding agents is that the incidence of false negatives for epithelial cells is minimized, and the visible staining reactions are generally stronger when the combined binding reagents are used in lieu of a single binding reagent.

Although the binding reagents and other reagents identified in the Examples are the preferred reagents for use in the context of the invention, the invention is intended to encompass epithelial-specific binding and staining reagents generally. These include, without limitation: Boehringer-Mannheim AE1 anti-cytokeratin antibody; Boehringer-Mannheim AE3 anti-cytokeratin antibody; Boehringer-Mannheim AE1/AE3 anti-cytokeratin antibody (AE1 and AE3 in admixture); Becton-Dickinson CAM 5.2 antibody, DAKO EMA antibody, Biomeda's Anti-Cytokeratin Cocktail CK22, Biomeda's Anti-Cytokeratin Cocktail CK23, Biomeda's Anti-Pan-Cytokeratin CK56, Biomeda's polyclonal goat or rabbit anti-cytokeratin antisera, ScyTek Laboratories' anti-EMA antigen antibody clone E29, and many others. Those skilled in the art and in possession of the guidance provided herein can readily determine alternative, equivalent binding and staining reagents and cocktails, to accomplish the disclosed result. These binding agents and cocktails may be used in combination with any known visualization system, such as the streptavidin, fluorescein- and digoxigenin-conjugated systems identified above.

As a control, Vimentin antibody is used as a binding alternative either in conjunction with binding and staining of the test cells, or subsequently thereto. In the context of this invention, Vimentin can be considered a binding reagent which is specific to non-epithelial cells.

The following examples are illustrative.

### Example 1

A tumor biopsy of approximately 100 mg of non-necrotic, non-contaminated tissue was harvested from the patient by surgical biopsy and transferred to the laboratory in a standard shipping container. Biopsy sample preparation proceeded as follows. Reagent grade ethanol was used to wipe down the surface of a Laminar flow hood. The tumor was then removed, under sterile conditions, from its shipping container, and cut into quarters with a sterile scalpel. Using sterile forceps, each undivided tissue quarter was then placed in 3 ml sterile growth medium (Standard F-10 medium containing 17% calf serum and a standard amount of Penicillin and Streptomycin) and was systematically minced by using two sterile scalpels in a scissor-like motion. The tumor particulates each measured about 1 mm³. After each tumor quarter was minced, the particles were plated in culture flasks using sterile pasteur pipettes (9 explants per T-25 or 20 particulates per T-75 flask). Each flask was then labeled with the patient's code, the date of explanation and any other distinguishing data. The explants were evenly distributed across the bottom surface of the flask, with initial inverted incubation in a 37° C. incubator for 5-10 minutes, followed by addition of about 5-10ml sterile growth medium and further incubation in the normal, non-inverted position. Flasks were placed in a 35° C., non-CO₂ incubator. Flasks were checked daily for growth and contamination. Over a period of a few weeks, with weekly removal and replacement of 5ml of growth medium, the explants grew out into a monolayer.

The cells were subsequently removed from the culture flask and a cell pellet was prepared by centrifugation. The cell pellet derived from the monolayer was then suspended in 5ml of the growth medium and mixed in a conical tube with a vortex for 6 to 10 seconds. The tube was then rocked back and forth 10 times. A 36 μl droplet from the center of the conical tube was pipetted onto one well of a 96 well plate. A fresh pipette was then used to pipette a 36 μl aliquot of trypan blue solution, which was added to the same well, and the two droplets were mixed with repeated pipette aspiration. The resulting admixture was then divided between two hemocytometer chambers for examination using a standard light microscope. Cells were counted in two out of four hemocytometer quadrants, under 10x magnification. Only those cells which had not taken up the trypan blue dye were counted. This process was repeated for the second counting chamber. An average cell count per chamber was thus determined. Using means known in the art, the quadrant count values were checked, logged, multiplied by 10⁴ to give cells/ml, and the total amount of fluid (growth medium) necessary to suspend remaining cell aliquots was calculated accordingly.

After the desired concentration of cells in medium was determined, additional cell aliquots from the monolayer were suspended in growth medium via vortex and rocking, and were loaded into a Terasaki dispenser known in the art. Aliquots of the prepared cell suspension were delivered into the microtiter plates using Terasaki dispenser techniques known in the art. A plurality of plates were prepared from a single cell suspension. Plates were then wrapped in sterile wet cotton gauze and incubated in an incubator box overnight, after which the microtiter wells were examined to assure that the cells had settled onto and adhered to each well base.

One microtiter plate was selected and segregated from the others intended for cytotoxicity assay, and the cells were examined as follows. Each well was overlaid with an aliquot of 95% ethanol for five minutes, 70% ethanol for five minutes, and 30% hydrogen peroxide/70% methanol for thirty minutes. Each well was then rinsed in Tris-saline several times.

Saving the first and last rows in the multi-well plate as controls, the remaining wells were each inoculated with approximately 8 microliters of an antibody cocktail including 80 microliters CAM 5.2 antibody (Becton-Dickinson), 10 microliters of a 1:30 dilution of AE1/AE3 (Boehringer-Mannheim) and 10 microliters of a 1:10 dilution of EMA antibody (DAKO). The wells were incubated at room temperature for one hour and were then rinsed in several changes of Tris-saline. Using an LSAB (streptavidin) kit, about 8 microliters of the secondary reagent (in the DAKO LSAB2 kit, the secondary reagent is yellow) was added to each well, followed by a one hour room-temperature incubation and several rinses in Tris-saline. The tertiary reagent (pink) was then inoculated in approximately 8 microliter amounts to each well, followed by a one hour room-temperature incubation followed by rinsing. An overlayer of Tris-saline was retained in each well while fresh filtered diaminobenzidine tetrahydrochloride (DAB)/H2O2 solution was prepared. The Tris-saline was discarded from each well and replaced with DAB solution. After a five minute room-temperature incubation, the cells were rinsed in distilled water, stained in hematoxylin for between 30 seconds and 1 minute, rinsed in warm running tap water for 2-5 minutes and rinsed in distilled water.

In the rows saved for use as a control, the above protocol was repeated except that the antibody cocktail was replaced with 8 microliters 1:200 dilution Vimentin antibody, with every other step being the same.

For the test wells, a strong brown stain upon visual inspection confirmed the identity of the cells as epithelial cells. Consistent with this observation, all the wells in the control rows were negative.

### Example 2

Example 1 was repeated except that in the test wells an alternate cocktail of binding reagents was used containing 60 microliters CAM 5.2, 20 microliters of a 1:30 dilution of AE1/AE3, and 20 microliters of a 1:10 dilution of EMA antibody. The test wells all stained positive (brown) for epithelial cells; the control wells were negative for non-epithelial cells.

Although the present invention has been described with respect to specific materials and methods above, the invention is only to be considered limited insofar as is set forth in the accompanying claims. For example, although solid tumors have been discussed above, any malignant cells including but not limited to blood, lymph and other cells may be subjected to the present protocols.

We claim:

1. A method for assessing sensitivity of patient cells comprising the steps of:
a) obtaining a malignant tissue specimen;
b) separating said specimen into multicellular particulates;
c) growing a tissue culture monolayer from said cohesive multicellular particulates;
d) inoculating cells from said monolayer into a plurality of segregated sites;
e) binding and staining some of said plurality of sites with a staining protocol including at least a staining protocol including one binding agent specific for epithelial cells;
f) treating the remainder of said plurality of sites with at least one agent;
g) examining said plurality of sites; and
h) assessing sensitivity of the cells in said remainder of said plurality of sites.

2. The method according to claim 1 wherein said plurality of segregated sites comprise a plate containing a plurality of wells therein.

3. The method according to claim 2 wherein said binding agent specific for epithelial cells includes at least one of anti-cytokeratin antibody and anti-epithelial-membrane-antigen antibody.

4. The method according to claim 3 wherein said binding agent specific for epithelial cells includes both anti-cytokeratin antibody and anti-epithelial-membrane-antigen antibody and further wherein the binding resulting from the addition of the binding agent is made visible via a chemical reaction with a streptavidin/peroxidase conjugate and a chromagen.

5. An antibody cocktail containing at least two monoclonal antibodies specific to cytokeratin and at least one monoclonal antibody specific to epithelial membrane antigen.

6. An antibody cocktail containing 80 microliters CAM 5.2 antibody (Becton-Dickinson), 10 microliters of a 1:30 dilution of AE1/AE3 (Boehringer-Mannheim) and 10 microliters of a 1:10 dilution of EMA antibody (DAKO).

7. An antibody cocktail containing 60 microliters CAM 5.2 antibody (Becton-Dickinson), 20 microliters of a 1:30 dilution of AE1/AE3 (Boehringer-Mannheim), and 20 microliters of a 1:10 dilution of EMA antibody (DAKO).

8. A method of confirming the epithelial character of malignant cells grown in culture, for use in a subsequent sensitivity assay, comprising binding a sample of said malignant cells with at least one binding agent specific for epithelial cell markers, conducting a chemical reaction with said binding reagent to render visible any resultant binding and to confirm the epithelial character of the cells, and subjecting fresh samples of the same malignant cells to sensitivity assays in vitro.

9. A method of confirming the epithelial character of malignant cells grown in culture, for use in a subsequent sensitivity assay, comprising binding a sample of said malignant cells with at least one binding agent further comprising an antibody cocktail containing at least two anti-cytokeratin monoclonal antibodies and at least one anti-epithelial membrane antigen monoclonal antibody, conducting a chemical reaction with said binding reagent to render visible any resultant binding, and subjecting fresh samples of the same malignant cells to sensitivity assays in vitro.

10. A method of confirming the epithelial character of malignant cells grown in culture, for use in a subsequent sensitivity assay, comprising binding a sample of said malignant cells with at least one binding agent further comprising an antibody cocktail containing 80 microliters CAM 5.2 antibody (Becton-Dickinson), 10 microliters of a 1:30 dilution of AE1/AE3 (Boehringer-Mannheim) and 10 microliters of a 1:10 dilution of EMA antibody (DAKO), conducting a chemical reaction with said binding reagent to render visible any resultant binding, and subjecting fresh samples of the same malignant cells to sensitivity assays in vitro.

11. A method of confirming the epithelial character of malignant cells grown in culture, for use in a subsequent sensitivity assay, comprising binding a sample of said malignant cells with at least one binding agent further comprising an antibody cocktail containing 60 microliters CAM 5.2 antibody (Becton-Dickinson), 20 microliters of a 1:30 dilution of AE1/AE3 (Boehringer-Mannheim), and 20 microliters of a 1:10 dilution of EMA antibody (DAKO), conducting a chemical reaction with said binding reagent to render visible any resultant binding, and subjecting fresh samples of the same malignant cells to sensitivity assays in vitro.
